(19) United States
(12) Patent Application Publication
(10) Pub. No.: US 2009/0254179 A1
(43) Pub. Date: Oct. 8, 2009

(54) METHOD AND APPARATUS FOR MINIMALLY INVASIVE IMPLANTS

(75) Inventor: Daniel R. Burnett, San Francisco, CA (US)

Correspondence Address: LEVINE BAGADE HAN LLP, 2400 GENG ROAD, SUITE 120, PALO ALTO, CA, 94303, US

(73) Assignee: NOVALERT, INC, San Francisco, CA (US)

(21) Appl. No.: 12/374,923

(22) PCT Filed: Jul. 24, 2007
(86) PCT No.: PCT/US2007/074258

§ 371 Date: Jun. 16, 2009

Related U.S. Application Data
(60) Provisional application No. 60/832,768, filed on Jul. 24, 2006.

(51) Int. Cl. A61F 2/12

(52) U.S. Cl. 623/8

(57) ABSTRACT

The present invention involves a device and method capable of providing minimally invasive insertion of implants with saline, aqueous or other fluid fillers while preventing deflation and/or migration, as well as monitoring for leakage from, or leakage into, implants (such as breast implants, pacemakers, implantable cardioverter defibrillators, other inflatable devices and other related devices). The device described herein has the ability to be inserted minimally invasively and to sense and communicate the occurrence of loss of integrity in the shell of virtually any implant.

### CROSS REFERENCE TO RELATED APPLICATION

Priority is claimed to U.S. Provisional Patent Application Ser. No. 60/832,768, filed Jul. 24, 2006, the contents of which are incorporated herein by reference.

### FIELD OF THE INVENTION

The present invention relates to the field of medical devices. In particular, the present invention relates to minimally invasive placement and monitoring of the integrity of liquid or gel-filled implants (such as breast implants) implanted within tissues or organs.

### BACKGROUND OF THE INVENTION

The primary parts of most breast implants are a shell (also known as an envelope or lumen), a filler, and a patch to cover a manufacturing hole. Breast implants may vary in shell surface (e.g., smooth or textured), shape (e.g., round or other shape), profile (i.e., how far it projects), volume, area, and shell thickness. With respect to the shell design, while most breast implants are single lumen (i.e., one shell), some breast implants are double lumen (i.e., one shell inside another shell). With respect to the filler, some breast implants are manufactured with a fixed volume of filler, some are filled during the implantation operation, and some allow for adjustments of the filler volume after the operation.

It should be noted that tissue expanders, which are silicone shells filled with saline, are regulated by FDA in a different way than breast implants. This is because tissue expanders are intended for general tissue expansion for a maximum of six months, after which, they are to be removed. Because of this, the design specifications (e.g., thinner shell) and preclinical testing recommendations are different for tissue expanders than for breast implants.

There are two basic types of gel in gel-filled breast implants: (1) silicone-gel implants and (2) hydrogel implants. While silicone gel implants are capable of maintaining their shape and have a convincingly realistic “feel” (particularly important for breast implants), they require chemical curing and exposure to toxic chemicals which requires them to be fully manufactured and inflated prior to insertion. Furthermore, exposure to these chemicals and materials constitutes a potential health hazard which resulted in the long-standing FDA ban on such materials in breast implants implemented in 1992 and still in effect. Furthermore, due to the radiographic nature of the silicone shell and the silicone gel, it is also notoriously difficult to detect ruptures in silicone-filled implants. In breast implants, for instance, the sensitivity and specificity of the costly, gold-standard, MRI are both approximately 75%. This means that one quarter of all patients will have an incorrect reading of the status of their silicone filled breast implant.

U.S. Patent Publication 2005/0267595 (published Dec. 1, 2005) describes a gastric balloon implantation device which includes as a leak monitoring system, a sensor that comprises a fine lattice or continuous film of detection material embedded in the wall or in between layers of the wall covering the entire device.

U.S. Patent Publication 2006/0111777 (published May 25, 2006) describes various implantation devices including breast implants which include as a leak monitoring system a sensor that comprises a fine lattice or continuous film of detection material embedded in the wall or in between layers of the wall covering the entire device.

### SUMMARY

Provided are devices for minimally invasive delivery of implants and monitoring for the integrity of implants, such as breast implants, that function to alert the user or a healthcare provider that the integrity of the implant is failing. Methods of using these devices also are provided. The devices are useful for reducing surgical incisions, reducing recovery time and measuring leakage into or out of the implant as well as other parameters such as changes in electrical properties within the device.

One embodiment of the device includes hydrogel which will greatly decrease exposure to foreign materials (many hydrogels are over 90% water or saline) in the event of a rupture. The hydrogels of the present invention require a minimum of 80% water/saline in their hydrated state in order to allow for sufficient compression to provide a minimally invasive state. The hydrogel may be manufactured and compressed from its final, solid, hydrated three-dimensional form (similar to a compressed cellulose sponge) and/or may be a semisolid viscous gel similar in texture to silicone oil, then desiccated or dehydrated in order to provide for compression prior to delivery. The solid, cohesive hydrogel which consists of hydrophilic groups covalently bonded to each other, may be free-floating or preferably, bonded to the shell of the implant. Alternatively, the shell of the device may be formed by dipping the preformed expanded hydrogel into a silicone liquid or casting the shell around the preformed hydrogel. The hydrogel used in the present invention may include, but should not be limited to: Chitin, Mannuronic Acid, Guluronic Acid, Glucomannan, Hyaluronic acid, Chitosan, PEG, HPMC, Pluronic, PVA and/or glycerol derived polymers. Alternatively, the hydrogel may also consist of acrylate, polyethylene oxide, cellulose, collagen, acetic acid, or other polysaccharide derived polymer. Alternatively, the polymer may be any hydrophilic, biocompatible material capable of being compressed and expanded upon rehydration. In further alternative embodiments, the hydrogel may consist of coating on the inside of implant shell and/or a coating on a silicone support structure within the shell which provides a hydrophilic interface to the silicone supports or silicone shell.

Use of the hydrogel of the present invention allows for the possibility of inflation or expansion in situ due to the possibility of desiccation pre-implantation and hydration post-implantation. This is a key feature and an important aspect of the present invention in that the rehydration in situ allows for the implant to be packaged within an insertion pod and placed minimally invasively through a small incision in the umbilicus or lower breast much as is done with saline-filled implants today. Unlike saline-filled implants today, though, the hydrogel implants of the current invention have a far superior feel, comparable to that of silicone-gel filled implants. Thus, through desiccation, compression, delivery, and rehydration of a hydrogel within a breast implant shell, the present invention provides the benefits of silicone-gel filled breast implant with the lower risks associated with a saline-filled implant. In its preferred embodiment, the hydrogel is created in its final shape covered by or inserted into a silicone shell and then desiccated and/or compressed after the patch, with rupture sensing capabilities as described below, affixed to the patch of the implant. This polymerization of the hydrogel followed by desiccation and cross-linking allows for a defined shape to the breast implant, a feature that is currently considered a main advantage of silicone implants over saline implants. Once the device is inserted into the body in its compressed state, it is then rehydrated using a water-based solution designed to create an isoosmolar environment within the implant. After rehydration, the filling line is removed from the implant and it assumes its final configuration.

The isoosmolar nature of the device is preferable if it is fully expanded, but another aspect of the invention provides for gradual expansion of the device in situ through the placement of a hyperosmolar solution or gel within the implant. This will function to slowly draw fluid into the implant (across the shell) and may provide for gradual device expansion until an iso-osmotic state is achieved. This strategy may be used to decrease the trauma of implantation through the reduction in surgical pocket formation, or may be used to create an optimal tissue expander. In this latter embodiment, the device is capable of providing gradual tissue expansion without the requirement for repeat clinic visits for needle puncture and inflation found in the current state of the art tissue expanders. The total expanded state could be tailored based on the solution infused into the implant and the solubility of the salts used may be such that relatively consistent, but never excessive, pressure is applied to the surrounding tissues.

Alternatively, the hydrogel implant of the present invention may provide for polymerization and/or gelling within the body once one or more solutions have been infused into the compressed and deflated implant. In this embodiment, the hydrogel need not be preformed and may instead be formed within the body. This embodiment has the added advantage of following dissection planes within the body, as well, and a potentially more natural look and feel. This embodiment may include a component of the hydrogel which may be inserted with the silicone shell and another component which is infused once the device is in place. In its optimal embodiment, this design may include completely safe and biocompatible materials which achieve their desired consistency via ionic cross-linking. An added advantage of ionic cross-linking is that the device may then be removed through breaking the ionic bonds and compression of the device.

Alternatively, the device may be a unique combination of pre-filled and injection filled due to unique properties of its design. For example, the device could be pre-filled with a certain amount of hydrogel, which may be bonded to the silicone shell of the implant, with the remaining hydrogel, saline or other aqueous solution infused at the time of placement. This embodiment is optimal for hydrogels that require partial hydration to maintain their structure, but which may then be further hydrated, or cross-linked, once the device has been placed in its implant pocket.

Further alternatively, the device may consist of 1) a fully-cured silicone shell, 2) a fully cured, lower durometer silicone or, preferably, a silicone foam and 3) saline which may be injected at the time of implantation. In this embodiment, a low-density, open-cell silicone foam may be present as a layer bonded to the inside of the shell (like a rind) or throughout the entire interior of the device. In combination with an iso-osmotic aqueous filler, for example saline or a liquid hydrogel, this open-cell foam may provide the feel of a silicone-gel implant with the established minimal risk safety profile of a saline implant. Furthermore, the silicone foam may allow for compression of the implant to allow for minimally invasive implantation reducing incision size, recovery time and surgical risk. The expansion of the shape-memory silicone foam, then, would also serve to more rapidly fill the implant to its desired level and prevent deflation in the event of a rupture of the outside shell. In combination with the rupture detection device and method described below, this embodiment of the invention will provide an unparalleled safety profile while also providing the feel and aesthetic effect of a silicone-gel filled implant. This embodiment, as well as the others mentioned above, may also be removed minimally invasively as described below.

In any of the embodiments of the current device, though, the device may also be removed minimally invasively through puncture of the device and exposure of the hydrogel to compounds designed to degrade the gel and/or mechanical forces, such as vacuum or agitating members, to break apart and/or evacuate the hydrogel. This is a key feature of the current invention in that minimally invasive removal upon rupture detection, as described below, will be required to maintain the aesthetic effect and minimization of surgical scar that is accomplished with insertion of the device. In the silicone foam embodiment described above, the device simply requires application of internal vacuum in order to compress the device for simple, minimally invasive extraction.

The hydrogel implant of the present invention may also allow for rapid, cost-effective, convenient and highly accurate detection of implant rupture. This feature is specifically enabled in hydrogel-filled, or other hydrophilic fluid-filled, implant of the present invention in that the conductive filler provides for a simple, low-profile addition to the breast implant patch (the strongest portion of the device) to provide a durable mechanism for accurate detection of implant rupture through detection of an abnormal conduction pathway. In particular, the patch need simply incorporate a small, externally or internally powered electronic chip which detects the absence or presence of an open circuit across the implant shell. An open circuit indicates that the highly insulating silicone shell is intact. A closed circuit, on the other hand, indicates that there is a conductive pathway across the insulating shell allowing current to flow through the hydrogel, across the shell, through the tissues surrounding the implant and then back to the sensing contact on the opposite side of the patch. With use of a relatively non-conductive fluid, with resistances of greater than 10 kOhm/cm, greater than 1 MOhm/cm and/or greater than 100 MOhm/cm, for example, a simple, fully internalized circuit will suffice. In this embodiment, upon rupture of the shell, the less conductive internal fluid is contaminated by the more conductive saline fluid (which tracks easily through the hydrophilic milieu) which can be easily detected by a fully internal circuit with both electrodes within the implant. This embodiment has the added advantage of not having to have a conducting path spanning the shell and possibly altering its long-term integrity. If a hydrophilic filler that is less conductive than saline is used, the previous mechanism of detecting a conducting path from the filler past the rupture in the shell and through the external implant pocket would also be effective in that the filler and shell would be relatively and extensively insulating, respectively. A breach in the shell would decrease the resistance path due to loss of the insulating silicone layer and, over time, the influx of saline will further decrease the resistance path leading to an even bigger change in resistance that may be easily detected. A further enhancement to this system would be the addition of a non-conductive material to the outside of the implant, i.e. titanium dioxide, to increase the resistance of the shell even further. Thus, a rupture of the shell would result in the loss of two heavily insulating layers, silicone and titanium resulting in a larger change in the resistance detected. In each of these embodiments, an abnormal conducting pathway may be reported via a variety of mechanisms including vibratory, acoustic, visual stimuli, EMF, radio or other signal to an external device.

While most hydrogels are superior to silicone with respect to biocompatibility, rupture detection is still important to reduce exposure of the patient to the implant filling material. With silicones, this exposure has been shown to create local inflammation and scarring, sometime severe enough to necessitate mastectomy, and silicones are suspected to cause other, more systemic problems as well. Exposure to biocompatible hydrogels are of less concern than chronic exposure to silicone due to the reduced total exposure (hydrogels are typically over 90% water) and typically less local scarring and inflammation. If the shell of the implant ruptures, though, exposure of the hydrogel has been found to cause some problems, such as calcification of the hydrogel within the implant (along with the associated difficulty in interpreting mammograms) making it prudent to ensure rupture detection and device replacement. Using the above-mentioned features of the device, an implant with a desirable look and feel may be placed minimally invasively, continuously monitored for rupture and removed minimally invasively in the event of a rupture while ensuring that the patient is never exposed to materials as inflammatory and potentially damaging as silicone.

The implant of the present invention may, also, be preferably coated with a highly biocompatible material, i.e. titanium, gold, PTFE, ePTFE, etc. to reduce the incidence of capsular contracture. In particular, the device of the present invention contemplates the use of a thin layer of ePTFE on the outside of the shell. This layer of ePFTE will serve multiple purposes, but its main function will be to encourage controlled ingrowth of bodily tissues to prevent capsular contracture. By allowing the cells of the body to be incorporated into the implant to some degree, the ePTFE layer will prevent the development of capsular contracture which occurs with use of a material (such as silicone) that prevents any tissue ingrowth and is therefore walled off by the body, in some cases providing tightness and pain around the implant. The thin coating of ePTFE may allow enough ingrowth to prevent capsular contracture, but resist intense ingrowth so as to allow for easy removal of the implant. This ePTFE coating (with an optimal internodular distance (“IND”) between 2 microns and 200 microns) will, ideally, not interfere with the stretch of the device and will be added after the shell has been manufactured.

As used herein, the term “lumen” refers to a cavity that is present inside of the shell of an implant. Also, as used herein, the term “patch” refers to a plug for an inflation or hydration opening of an implant, which plug generally defines a discrete region of increased durometer and/or thickness through which the implant may be inflated or filled. The inflation patch is typically formed from a thicker and stronger silicone than the rest of the shell and is added, usually by vulcanization, to the remainder of the implant shell after the shell has been fully manufactured.

These and other features, aspects, and advantages of the present invention will become more apparent from the following description, appended claims, and accompanying exemplary embodiments shown in the drawings.

### BRIEF DESCRIPTION OF THE DRAWINGS

FIG. 1A is a cross-section of the circumferential hydrogel or silicone foam embodiment in which a hydrogel or silicone foam layer may be incorporated beneath the external shell of the device;

FIG. 1B is a cross-section of the hydrogel or silicone foam embodiment in which a hydrogel or silicone foam fills the entire cavity of the device;

FIG. 2 is a cross-section of a hydrogel or silicone support embodiment in which the silicone foam, low-durometer silicone or hydrogel form struts or supports within the implant;

FIG. 3A-C are cross-sections of the compressed devices from FIGS. 2 and 3 as well as an empty external shell ready for implantation;

FIG. 4 is a cross-section of the deployment of a hydrogel or silicone foam embodiment in which a hydrogel or silicone foam fills the entire cavity of the device and is expanded or rehydrated once it has been implanted in the body;

FIG. 5 is a cross-section of the deployment of a circumferential hydrogel or silicone foam embodiment in which a hydrogel or silicone foam layer may be incorporated beneath the external shell of the device and is expanded or rehydrated once it has been implanted in the body;

FIG. 6 is a cross-section of the deployment of a hydrogel embodiment in which the hydrogel is cross-linked upon injection into the implant or assumes its shape-memory configuration once it has been inserted within the shell of the implant; and

FIG. 7A-C are perspective views of the internal sensor and internal/external sensor of the present invention wherein the internal sensor, FIG. 7B, may be used if the iso-osmotic filling material has measurable properties (electrical properties, pH, etc.) different from saline and the internal/external design, FIG. 7C, may be used to detect an electrical connection across the insulating silicone shell if the device filling is saline or has similar measurable properties (electrical properties, pH, etc.).

FIGS. 8A-B are perspective views of the fully cured silicone lattice embodiment illustrated inside of the perforating fixture and post-perforation.

### DETAILED DESCRIPTION

Presently preferred embodiments of the invention are illustrated in the drawings. An effort has been made to use the same, or like, reference numbers throughout the drawings to refer to the same or like parts.

The proposed implant monitoring device of this application serves as a solution to the issues of: 1) Providing minimally invasive insertion of implants with saline or aqueous fluid fillers while preventing deflation and/or migration, and 2) Monitoring for leakage from, or leakage into, implants (such as breast implants, pacemakers, implantable cardioverter defibrillators, other inflatable devices and other related devices). The device described herein has the ability to be inserted minimally invasively and to sense and communicate the occurrence of loss of integrity in the shell of virtually any implant.

The competitive advantages of the present invention include minimally invasive insertion with a safe aqueous filler while providing the feel of the less safe silicone-filled implants. In addition, the integrity monitoring system of the present invention provides: (1) continuous (or intermittent but frequent) monitoring of implant integrity; (2) an implant failure signaling mechanism for both the patient and healthcare professional; and (3) the ability to have a sensor communicate, with an external device, information about the state of an implanted device.

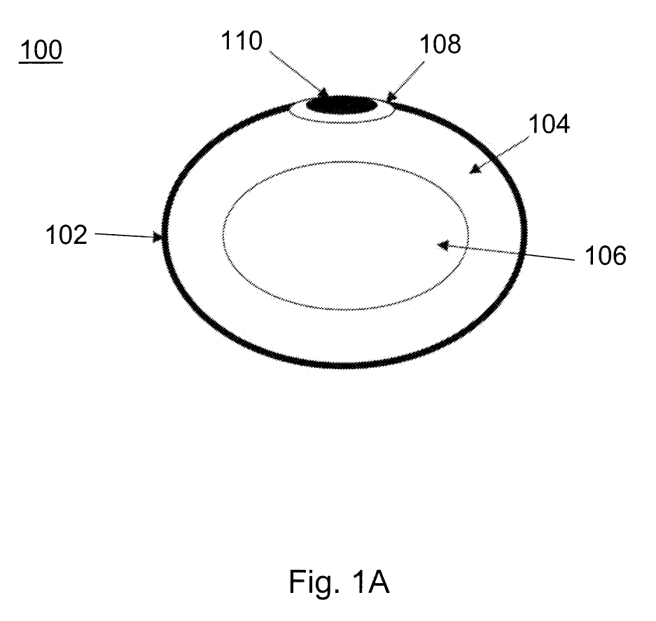

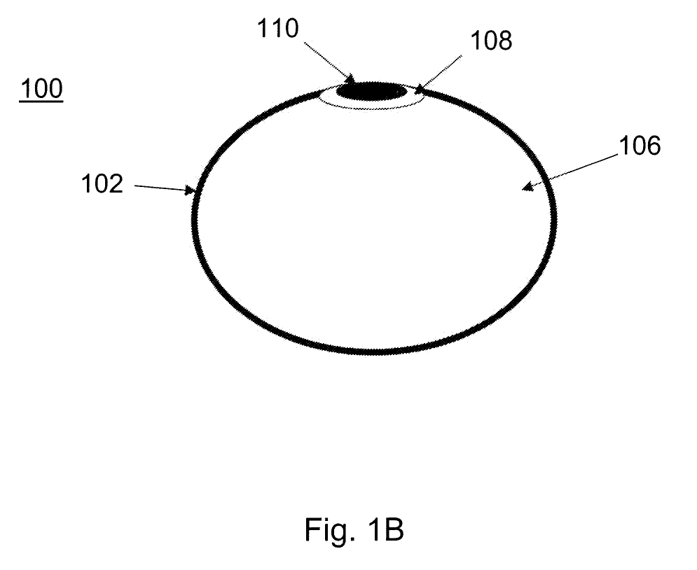

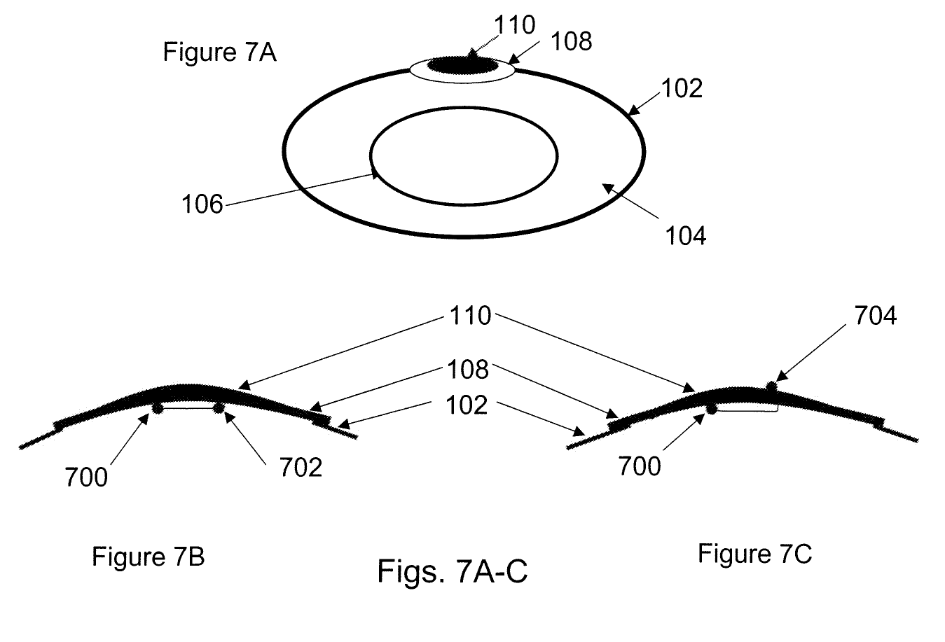

FIG. 1A is a cross-section of the circumferential hydrogel or silicone foam embodiment of the present invention 100 in which a hydrogel or silicone foam layer 104 may be incorporated beneath the external shell 102 of the device. In this embodiment, the silicone foam or hydrogel layer 104 within the implant provides two functions: (1) it enables the implant to be compressed prior to insertion and expanded upon implantation and (2) it helps maintain the shape of the implant even when partially filled. In this silicone foam embodiment, the foam may preferably be an open-cell foam to allow for a large degree of compression of the foam prior to insertion and may consist of foams of varying durometers, strength and porosity. In addition, the silicone foam within a silicone implant may consist of different types of foam within a single implant to give the appropriate mechanical properties. For example, a softer foam with a lower density of silicone and larger pores may be directly adjacent to the shell of the implant to allow a softer, more gelatinous feel while a firmer foam may be used more centrally to provide more bulk. Any combination of foams may be used though, to tailor the desired look, feel and function of the implant. The central region 106 of the implant illustrated in this figure may be of any size ranging from the bulk of the diameter of the implant (with just a thin coating of foam 104 inside the shell 102) to a small central pore within the device. In the hydrogel embodiment, the foam of the central region 106 described above may be replaced with a partially or fully dehydrated hydrogel which may be rehydrated upon insertion of the device. Alternatively, the hydrogel may be fully hydrated upon insertion and only the central aperture may be filled with a hydrophilic or hydrophobic fluid. In the hydrogel embodiment, in particular, a combination of a hydrophobic filler (i.e. an oil) and a hydrophilic filler (saline, water or a fluid hydrogel) may be used due to the hydrogel preferentially drawing in the aqueous fluid and allowing for distinct fluid accumulations in each compartment. In either the hydrogel or the silicone foam embodiments, the foam or hydrogel may be bonded to the shell 102 of the device or may be free-floating. Both embodiments may incorporate the rupture sensor 110 within the patch 108 of the device, within a distinct region of the device shell 102 or within the hydrogel or foam filler itself. The rupture sensor 110 is further described in FIG. 7. One major advantage of this layer of foam and/or hydrogel 104 underneath the silicone shell 102, as well, is the avoidance of folding and creasing of the silicone shell 102. Of the devices that rupture within the patient, a large percentage of the ruptures occur due to folding of the thin silicone shell and the creasing and cracking that subsequently occurs. By adding a layer of lower-durometer silicone, silicone foam and/or hydrogel 104, the device of the present invention will greatly reduce the incidence of creasing due to the inability of the shell 102 to fold directly back on itself. The shell 102 may still form a fold on itself, but the layer of silicone or foam 104 will ensure that the shell 102 folds back on itself in less-damaging way due to the increase of the turn radius of the folded shell 102 from virtually 0 to a measurable distance which prevents creasing and loss of integrity.

FIG. 1B is a cross-section of the hydrogel or silicone foam embodiment of the present invention 100 in which a hydrogel or silicone foam fills the entire cavity 106, encompassed by the shell 102, of the device. In this embodiment, the central void 106 of the device 100 may be filled with hydrogel or silicone foam. In this embodiment, as well, foams and/or hydrogels of varying durometer and firmness may be used within a single device and both hydrophilic and/or hydrophobic filling solutions may be used in a single device, as well, to provide the optimal functional properties.

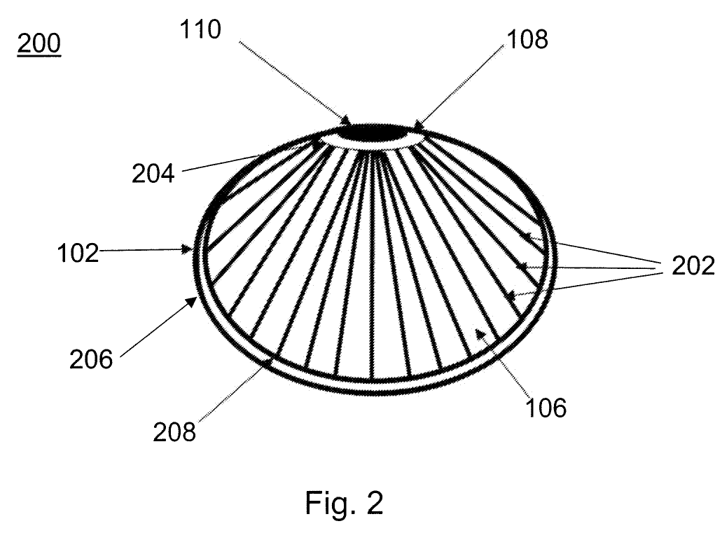

FIG. 2 is a cross-section of a hydrogel or silicone support embodiment 200 in which the silicone foam, low-durometer silicone or hydrogel form struts or supports 202 within the implant. In this embodiment, the hydrogel, silicone foam or fully cured solid silicone may form struts or other support configurations 202 to provide a more gelatinous feel to the surface of the filled device 200. In this embodiment, the struts or other support member or members 202 (i.e. a lattice, a cage, a series of columns, etc.) may be of varying firmness or durometer and may used in combination with hydrophilic and/or hydrophobic filling solutions 106. As illustrated in this figure, the support structure 202 preferentially spans the device and connects one section 204 of the device shell 102 to another section 206. In order to prevent erosion and rupture of the shell 102, the support structure 202 is preferentially bonded to the shell 102 and the shell 102 may be further reinforced at these contact points 204,206. In an optional embodiment of the invention, a hydrogel, silicone foam or low-durometer solid rim 208 encapsulates the lumen filled with hydrophilic and/or hydrophobic filling solutions 106.

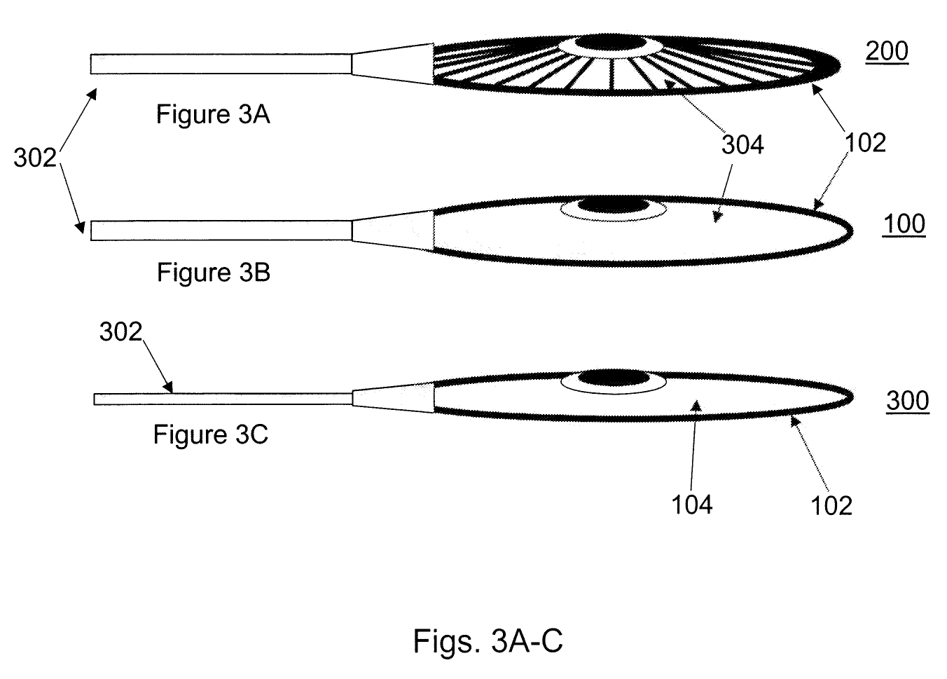

FIGS. 3A-C are cross-sections of the compressed devices 100, 200 from FIGS. 1 and 2 as well as an empty external shell 300 ready for implantation. In these figures, the various embodiments of the device are shown loaded for implantation within the patient. In this embodiment, the device may be deployed via expulsion from the delivery device 302 or the delivery device 302 may open (like a pod or a clam-shell) and the device may be released in this manner. In FIG. 3A, the silicone foam or hydrogel-filled embodiment 200 of FIG. 2 is shown in its compressed state prior to delivery. Upon delivery, the device 200 may be expanded via rehydration or release of vacuum and filled with a hydrophilic and/or hydrophobic filling solution 304. In FIG. 3B the device 100 of FIG. 1A may be expanded via rehydration or release of vacuum and filled with a hydrophilic and/or hydrophobic filling solution 304. In FIG. 3C, the device 300 may be filled and/or inflated after implantation. In this embodiment, the device 300 is delivered into the patient and then inflated with a filling medium which may cross-link within the patient or may form a gelatinous hydrogel mixture. Alternatively, a pre-formed hydrogel and/or foam-based material 104 may be inserted into the shell 102 of the device 300 after the device 300 has been placed within the implantation pocket. Separate insertion of these components provides for a smaller insertion incision while also providing the support and shape of a shape-memory material. In this embodiment, the foam or hydrogel 104 may be bonded to the shell 102 upon implantation or may float freely within the device 300.

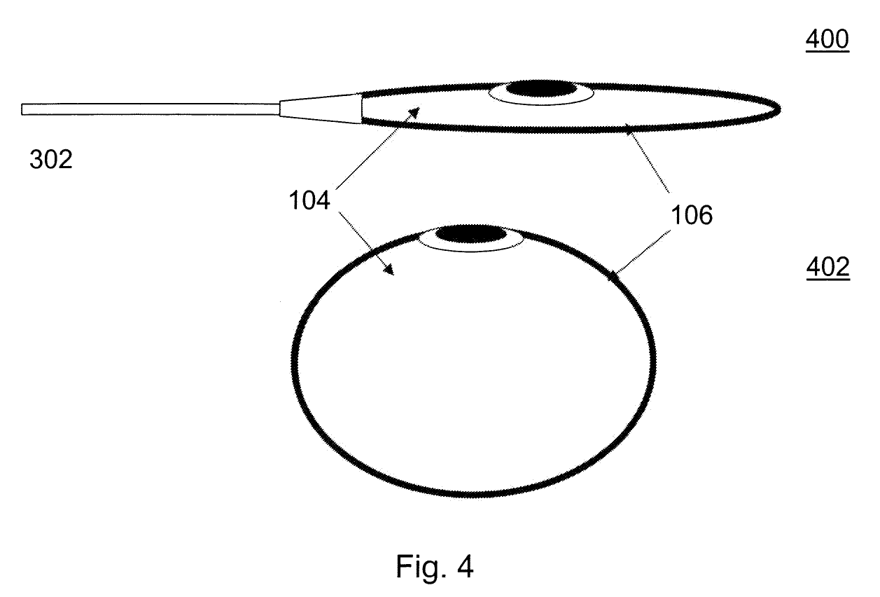

FIG. 4 is a cross-section of the deployment of a hydrogel or silicone foam embodiment in which a hydrogel or silicone foam 104 fills the entire cavity 106 of the device 100 and is expanded or rehydrated once it has been implanted in the body. In this figure, the device is shown in its compressed and/or dehydrated state 400 and in its expanded or rehydrated state 402. While the expansion and filling within the body is not a necessary prerequisite for the invention (for example, the silicone foam device may be pre-expanded and pre-filled with saline prior to insertion), this procedure will allow for decreased complexity and complications with the implantation procedure by allowing it to be less invasive. The device 400 may be inflated with hydrophilic and/or hydrophobic filling solutions 304 and may be filled to varying degrees, up to a specified maximum, based on the required tension and volume of the device 100.

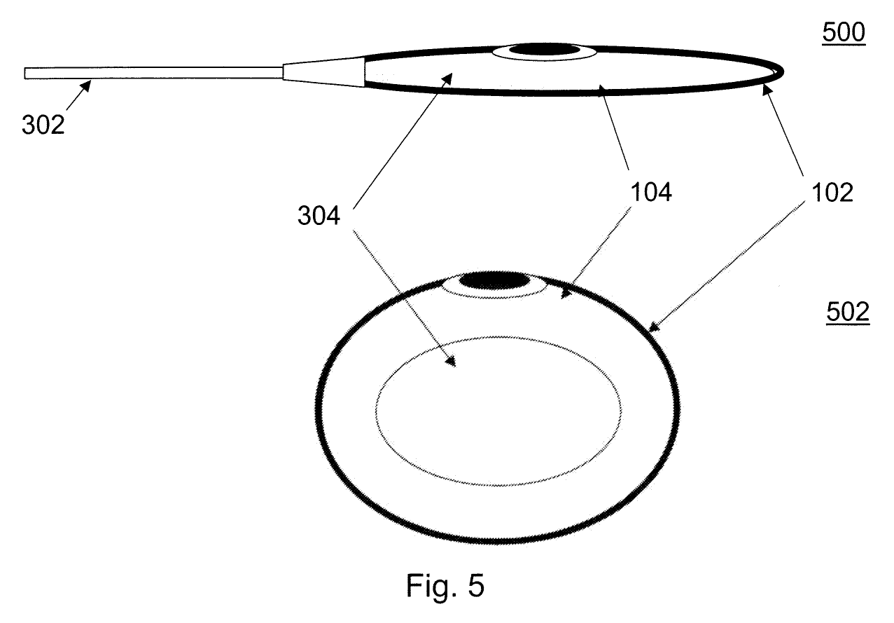

FIG. 5 is a cross-section of the deployment of a circumferential hydrogel or silicone foam embodiment in which a hydrogel or silicone foam layer 104 may be incorporated beneath the external shell 102 of the device and is expanded or rehydrated once it has been implanted in the body. In this figure, the device is shown in its compressed and/or dehydrated state 500 and in its expanded or rehydrated state 502. While the expansion and filling within the body is not a necessary prerequisite for the invention (for example, the silicone foam device 500 may be pre-expanded and pre-filled with saline prior to insertion), this procedure will allow for decreased complexity and complications with the implantation procedure by allowing it to be less invasive. The device may be inflated with hydrophilic and/or hydrophobic filling solutions 304 and may be filled to varying degrees, up to a specified maximum, based on the required tension and volume of the device. While the support layer 104 beneath the shell 102 is preferentially bonded to the shell 102 itself, it may also be free-floating.

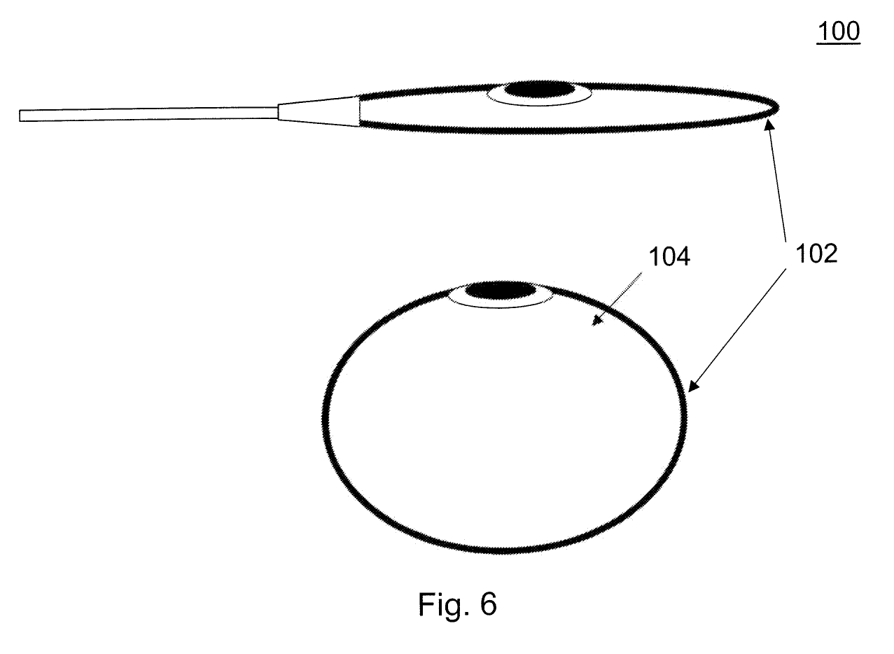

FIG. 6 is a cross-section of the deployment of a hydrogel embodiment in which the hydrogel 104 is cross-linked upon injection into the implant 100 or assumes its shape-memory configuration once it has been inserted within the shell 102 of the implant 100. In this embodiment, the device 100 may be inserted separately from its hydrogel or supportive silicone foam components 104 which may either cross-link after implantation or assume the shape from its previous cross-linking outside of the body. In the shape-memory embodiment, the foam or hydrogel 104 may assume a preconfigured shape once it is inserted within the shell 102 and may or may not bond to the shell 102, as well.

FIGS. 7A-C are perspective views of the internal sensor and internal/external sensor of the present invention. In FIG. 7A, the sensor 110 is incorporated into device 100 as part of the patch 108. The patch 108 permits closure of the shell 102. The sensor 110 may be internal or external to the shell 102 of the device 100 or a combination of the two. In FIG. 7B, an internal configuration of the sensor 110 is shown. The internal configuration may be used if the iso-osmotic filling material 104 has measurable properties (electrical properties, pH, diffusion, etc.) different from the saline 106. In FIG. 7C an internal/external combination of sensor 110 is shown. This internal external combination of sensor 110 may be used to detect an electrical connection across the insulating silicone shell 102 if the device filling 106 is saline or has similar measurable properties (electrical properties, pH, diffusion, etc.). In the event that the hydrogel and hydrophilic and/or hydrophobic filling solutions 304 vary significantly from the fluid found outside the implant 100 in the implant pocket in the sensed characteristic (pH, conductivity, protein concentration, etc.) a fully internal sensor 110 may be used if tracking of fluid 304 is expected within the implant 100. For example, if an aqueous filler 106 is used with a pH or conductivity that is greater or less than that of physiologic fluid in the implant pocket, a simple, fully internal sensor 110, as found in FIG. 7B, may be used to detect a change in pH or fluid conductivity. In the event, though, that the aqueous filler 106 is not significantly different from the fluid surrounding the implant in the sensed characteristic (in this case conductivity or other related electrical property) an internal/external sensor 110 may be required, as in FIG. 7C. In this embodiment, for example, a small voltage or current may be induced by the sensor 110 within the implant 100 and sent to the internal contact 702 inside of the patch 108 or the external contact 704 outside of the patch 108. The complementary electrical contact 700 may then be sensed to detect the presence of an abnormal conducting pathway. Due to the powerful insulation provided by the silicone shell 102, a conducting pathway should not be sensed if the shell 102 is intact. In the instance of a rupture, though, a conducting pathway will be present and the current or voltage will be sensed by the sensing electrical contact 700. This change in condition will then be transmitted to the sensor circuitry 110 and stored for communication to the healthcare professional or communicated to the patient or healthcare provider directly. The sensor may, preferentially, be radio frequency (RF)-enabled and the implant status may be communicated externally by RF or by other wireless technology.

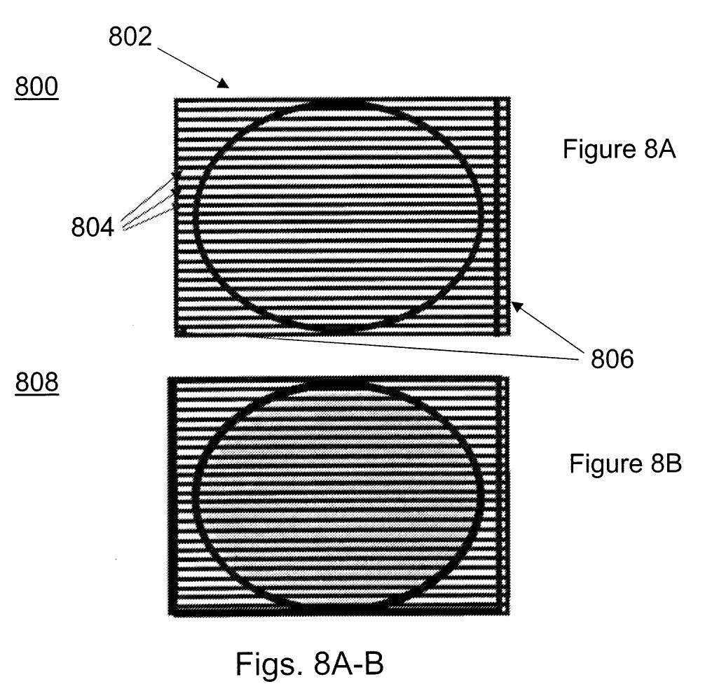

FIG. 8A-B are perspective views of the fully cured silicone lattice embodiment illustrated inside of the perforating fixture 800 and post-perforation 808. In this embodiment, the preferably three-dimensional silicone lattice may be formed by a specialized molding fixture 800 with rods 804 that slide into the mold 802 from one or more plate 806. The silicone lattice may then be cast into the mold 802 and the rods 804 removed once the silicone has fully or partially cured. Alternatively, the silicone lattice may be formed by molding a fixed shape out of silicone and then perforating the structure after the device has been removed from the mold 802. The perforations may be achieved through the use of lasers, drills, coring needles, or any other perforation generating mechanism. The liner perforations may be one, two or three-dimensional and may communicate fully or only at select points. The texture and flow of saline within the implant may be tailored by allowing for slower or more rapid flow from the linear perforations or, optionally, compartments within the breast implant.

The present invention has been envisioned as being highly useful for any inflatable implant, including breast implants, percutaneous gastrostomy tubes, Foley catheters, penile implants, gastric balloons, etc. Further, due to the relative ease of measuring electrical properties and relative ease of translation to an RF-based technology, the rupture sensing element could be reduced significantly in size or even simply encompass an RF and electrical property sensing element that are printed on the inside of the implant to be monitored. In this way, changes in electrical properties can be quickly and easily measured and reported in a very low-profile manner within the implant. This feature may also apply to other characteristics of the filling fluid including chemical, optical, physical, pH, electrical properties, etc.

While RF has been mentioned as a communicating mechanism, a variety of other mechanisms may be employed including auditory, acoustic, vibrational or other stimuli to alert the patient that the implant has been compromised. Also, while RF has also been mentioned as a method of powering or charging the device, the device may also be powered by alternative mechanisms, including a self-winding mechanism (as found in watches), an internal rechargeable battery, or a long-lasting capacitor/internal battery. These alternative charging and alerting mechanisms all provide for an additional safeguard in that the patient may be notified nearly instantaneously of a rupture and not require the additional step of exposure to an RF transmitting/receiving apparatus.

Furthermore, while symmetric embodiments of the present invention are illustrated in the attached figures, asymmetric embodiments have also been anticipated. In these embodiments, the shape of the breast may be more accurately represented by an implant that tapers from a narrow top to a more fuller base. These, and any, of the implants of the present invention may be used as breast lifts, breast augmentation, breast reconstruction or any other inflatable implant.

Lastly, while silicone foams and hydrogel have been described, any shape-memory or deformable material may be used so long as it may be compressed into a smaller format for insertion then expanded or inflated once it has been placed within the body.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

What is claimed is:

1. A device for minimally invasive implantation comprising:
a shell; and
a support material disposed within the shell, the support material being adapted to be compressed for implantation and subsequently expanded.

2. The device of claim 1, wherein the support material comprises a hydrogel or hydrophilic material.

3. The device of claim 2, wherein the hydrogel or hydrophilic material is dehydrated prior to implantation.

4. The device of claim 3, wherein the dehydrated hydrogel or hydrophilic material is hyper-osmotic so that it expands after implantation due to ingress of water through the shell from surrounding tissue.

5. The device of claim 1, wherein the shell is configured to be one of a breast implant, a pectoral implant, a calf implant, a bicep implant, a gluteal implant, a penile implant, a gastric implant, or a peritoneal implant.

6. The device of claim 1 further comprising a plurality of silicon supports.

7. The device of claim 1, wherein the support material comprises an open-celled silicon foam.

8. The device of claim 1, wherein the support material comprises a plurality of open-celled silicon foams of differing properties selected to replicate the external characteristics of a silicone-gel implant.

9. The device of claim 8 further comprising a plurality of silicon supports.

10. The device of claim 9, wherein the plurality of silicon supports form a lattice.

11. The device of claim 1 further comprising a lumen surrounded by the support material.

12. The device of claim 11 further comprising a hydrogel filling the lumen.

13. The device of claim 1, wherein the device is adapted to be inflated using air or water.

14. The device of claim 1 further comprising circuitry for detecting a breach in the shell.

15. The device of claim 14 further comprising a seal in the shell, wherein the circuitry is disposed in the seal.

16. The device of claim 14, wherein the circuitry further comprises circuitry for detecting a change in a chemical, physical, or electrical property sensed between at least two different locations.

17. The device of claim 16, wherein one of the at least two different locations is inside the shell and another of the at least two different locations is outside the shell.

18. The device of claim 16, wherein both of the locations are inside the shell.

19. The device of claim 16 further comprising communication circuitry.

20. The device of claim 19, wherein the communication circuitry comprises circuitry for providing a patient a self-detectable alert indicating a compromise of the shell.

21. The device of claim 19, wherein the communication circuitry comprises circuitry for communicating with a device external of the patient.

22. The device of claim 21, wherein the circuitry comprises radio circuitry.

23. The device of claim 22, wherein the radio circuitry comprises radio frequency identification circuitry (RFID).

24. The device of claim 1, wherein the support material comprises a silicon material having a plurality of interconnecting perforations.

25. A method for providing a self-expanding implantable a device:
providing an shell;
disposing within the shell a hyper-osmotic, dehydrated support material,
wherein the shell is hyper-osmotic so that after implantation, the support material absorbs water from surrounding tissue and expands.
